(19) United States
(12) Patent Application Publication
(10) Pub. No.: US 2007/0173745 A1
(43) Pub. Date: Jul. 26, 2007

(54) Rod length measuring instrument

(76) Inventors: Jennifer Diederich, Cumberland, RI (US); Noelle Dye, Charlestown, MA (US); Rob Brown, Lexington, MA (US)

Correspondence Address: CARR LLP (IST), 670 FOUNDERS SQUARE, 900 JACKSON STREET, DALLAS, TX, 75202, US

(21) Appl. No.: 11/676,101

(22) Filed: Feb. 16, 2007

Related U.S. Application Data
(63) Continuation-in-part of application No. 29/261,357, filed on Jun. 13, 2006.
(63) Continuation-in-part of application No. 10/990,272, filed on Nov. 16, 2004.

(51) Int. Cl. A61B 5/103

(52) U.S. Cl. 600/587

(57) ABSTRACT

There is disclosed an apparatus and method for measuring the distance between the bone anchors inserted into vertebrae comprising two legs pivotally coupled to each other, the two legs are also coupled to a proportional magnifier which is coupled to a scale. The proportional magnifier allows the scale to be easily viewable.

### CROSS-REFERENCE TO RELATED APPLICATIONS

This application is a Continuation-in-Part of prior commonly assigned design application Serial Number 29/261,357, “Rod Length Indicator,” filed Jun. 13, 2006, and a Continuation-in-Part application to prior commonly assigned application Ser. No. 10/990,272, “An Implant Assembly and Method for Use in an Internal Structure Stabilization System,” filed Nov. 16, 2004, currently pending, the entire contents of both applications are incorporated herein by reference for all purposes

### FIELD OF THE INVENTION

This disclosure relates to devices, instruments, apparatuses, and methods for stabilizing bony structures, more particularly, to devices, instruments, apparatuses, and methods for measuring the distance between bony structures.

### BACKGROUND

Patients suffering from orthopedic injuries, deformities, or degenerative diseases often need surgery to stabilize an internal structure, promote healing, or relieve pain. Surgeries to correct spinal problems often involve placing implants such as braces, rods, and various implants between one or more of the patient's vertebrae, anchored into the vertebrae pedicles by screws or hooks. Traditional surgical procedures to correct injuries, defects, and/or abnormalities of the spine have heretofore been substantially invasive. In addition to trauma to the nerves and tissue surrounding the incision, traditional invasive procedures pose significant risk of damage to vital intervening tissues and major muscles and ligaments of the back. The resulting trauma to the tissue and nerves generally requires long recovery periods for the patient and a significant amount of pain experienced during such recovery.

Recently, minimally invasive procedures and micro-surgical procedures have been developed for correction of spinal injuries, defects, and/or abnormalities. These procedures generally involve cutting a small channel down to the affected spinal area and inserting micro-surgical instruments including rod reduction devices into the channel or by using cannulas and the like for receiving instruments therein. Implant engaging instruments such as extensions from the implants may be used for adjustment and manipulation of the implants after the implants have been placed into the bony structures. These percutaneous, minimally invasive and micro-surgical procedures generally cause less disruption to surrounding and intervening tissues and muscles and therefore result in a quicker and less painful recovery period.

Many minimally invasive procedures are practiced for inserting spine stabilization systems to correct defects of the spine. Most spine stabilization systems require implanting bone anchors into vertebrae, the anchors thereafter accompanied by various components such as stabilizing medical implants, which may include rods, braces, connectors, and the like. Before implanting stabilizing components such as a rod, connector, and the like, the surgeon may need to measure the distance between the vertebrae in order to determine the correct size of implant required. Heretofore, available instruments have not been able to provide convenient measurements of the distance between the bone anchors at the point of insertion into the vertebrae when the measurements occur in a percutaneous manner.

Accordingly, what is needed is an instrument which can accurately measure the distance between two points along the spine such as the distance between bone anchors or pedicles in a percutaneous manner. Certain aspects of the present invention provide methods and apparatuses used in percutaneous and subcutaneous surgical techniques for correcting spinal defects and injuries.

### SUMMARY

There is disclosed an apparatus and method for measuring the distance between the bone anchors inserted into vertebrae comprising two legs pivotally coupled to each other, the two legs are also coupled to a proportional magnifier which is coupled to a scale. The proportional magnifier allows the scale to be easily viewable.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a perspective view of one embodiment of a measuring instrument;

FIG. 2 is a perspective view of the measuring instrument shown in FIG. 1 shown in a second position;

FIG. 3 is an enlarged perspective view of the feet of the measuring instrument shown in FIG. 1;

FIG. 4 is an enlarged perspective view of the proportional magnifier of FIG. 1;

FIG. 5 is a perspective view of one embodiment of a measuring instrument;

FIG. 6 is an enlarged perspective view of a distal end portion of the measuring instrument shown in FIG. 5;

FIG. 7 is a perspective view of one embodiment of a measuring instrument;

FIG. 8 a perspective view of the measuring instrument of FIG. 7 while in use; and

FIG. 9 is a perspective view of a kit for implanting a stabilization system.

### DETAILED DESCRIPTION

In the following discussion, numerous specific details are set forth to provide a thorough understanding of the present invention. However, those skilled in the art will appreciate that the present invention may be practiced without such specific details.

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

As used in this application, the term proximal refers to a portion closer or nearest to the user of an instrument and the term distal refers to a portion farthest from the user of the measuring instrument. The term end refers to the terminating portion of a component or any portion that is proximal to the terminating portion.

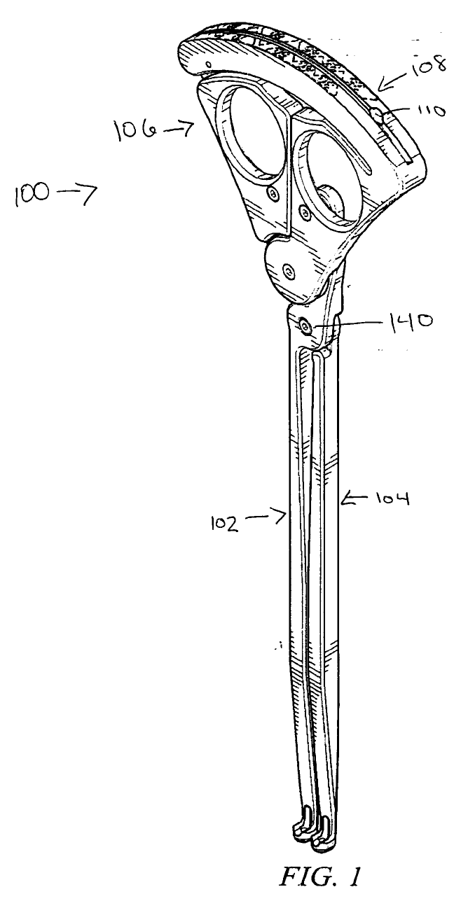

Referring now to FIG. 1, there is shown one embodiment of a measuring device 100 in a first position that may be used to measure the distance between two bony structures. The measuring device 100 may include a first leg 102 and a second leg 104 coupled to each other. In certain embodiments the first and second legs 102, 104 are pivotally coupled to each other. In some embodiments, the distal end of the first and second legs may be adapted to be placed adjacent a bony structure or a device that may be implanted into the bony structure. The proximal end portions of the first and second legs 102, 104 may be coupled to a proportional magnifier 106. The proportional magnifier 106 may be coupled to a scale 108 with a indicator 110 that indicates to a user the distance between the bony structures that may be adjacent the distal end of the first and second legs 102, 104.

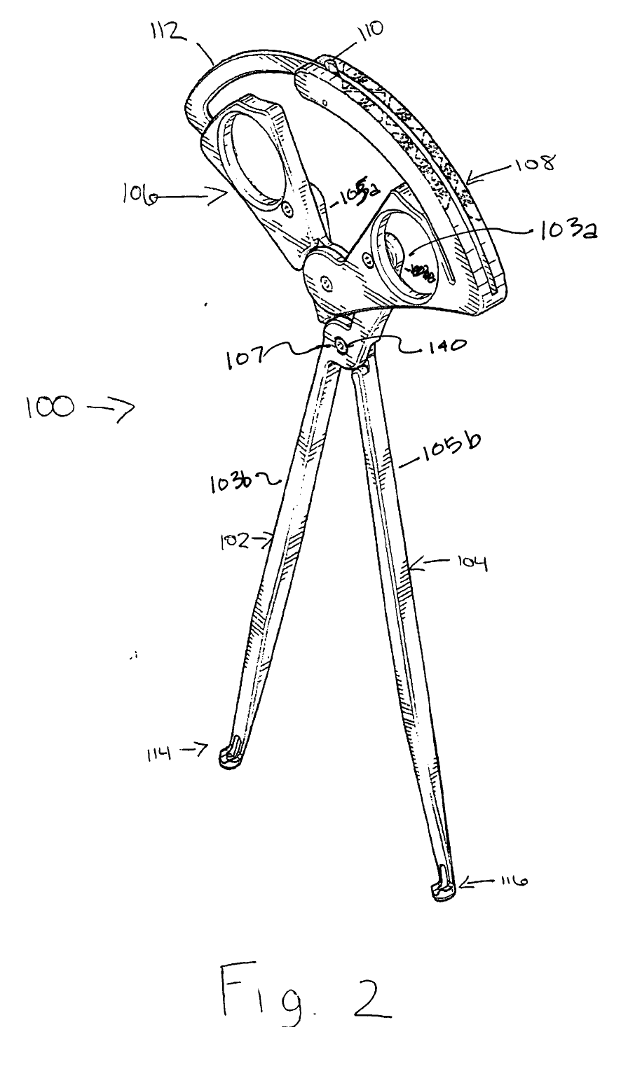

Turning now to FIG. 2, there is illustrated the measuring device 100 shown in a second position. The second position shows the measuring device 100 measuring the distance between two structures (not shown). In certain embodiments the indicator 110, which may be attached to an indicator arm 112, lines up with a marking on the scale arm 108 that corresponds to a distance. The distance that may be measured is the distance between a first distal end or foot 114 of the first leg 102 and a second distal end or foot 116.

As illustrated in this embodiment, the leg 102 can be divided into two portions a proximal portion 103a and a distal portion 103b. Similarly the leg 104 can be divided into a proximal portion 105a and a distal portion 105b. In certain embodiments, the legs 102 and 104 may be coupled to each other at a pivot point 107 with the pin 140. For percutaneous situations, the distal portions 103b and 105b may be relatively long in comparison to the proximal portions 103a and 105a. Thus, the pivot point 107 is relatively high to allow the ends 114 and 116 to reach the vertebra in a percutaneous manner. As illustrated, because the distal portions 103b and 105b are relatively long, the proximal portions 103a and 105a are relatively short to reduce the overall length of the instrument. As those skilled in the art would appreciate, if a scale were to be coupled directly to the proximal portions 103a and 105a, the scale would be relatively small and accurate measurements would be more difficult. So, in the illustrative embodiment, the proportional magnifier may be used to “increase” the size of the scale. The proportional magnifier 106, therefore, may also allow a user of a device to accurately determine the distance between the first and second feet 114, 116 without resorting to a conversion factor. In certain embodiments, the markings on the scale arm 108 may directly correspond to the distance between the feet 114, 116.

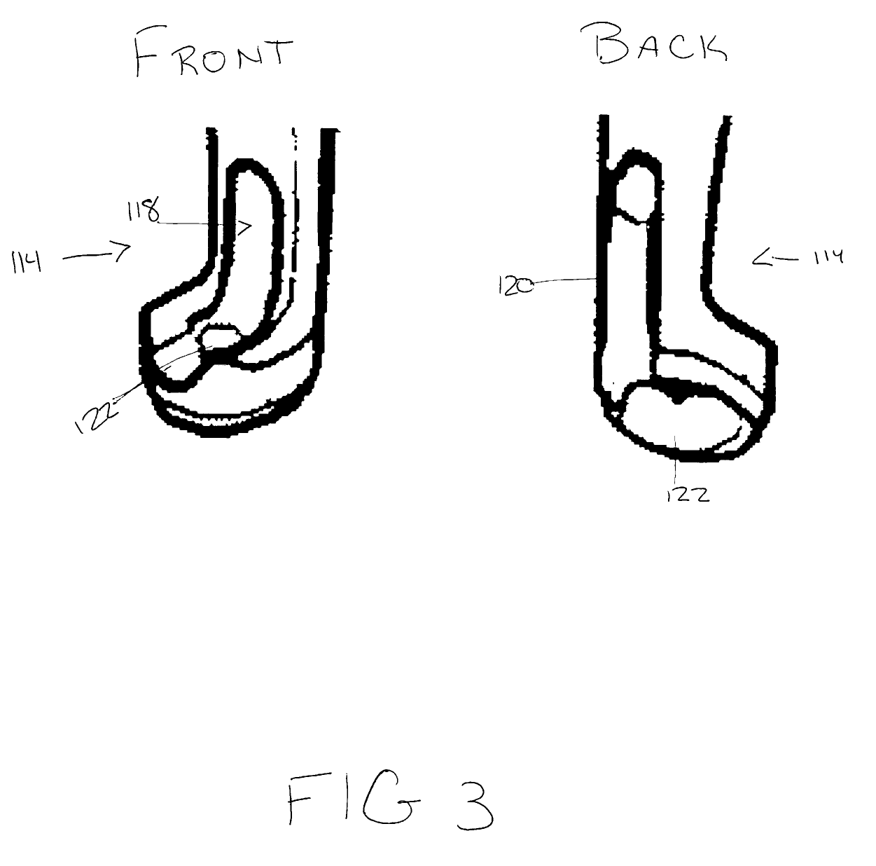

Referring now to FIG. 3, an enlarged view of the front and back of the first or second foot 114, 116 is shown. As shown, each foot may include a first elongated slot 118 on the front and a second elongated slot 120 on the back of the foot for engaging a guide wire (not shown). Each foot may also include an aperture 122 at the distal end for engaging a portion of the vertebra. The first and second elongated slots 118, 120 and the aperture 122 may cooperate to act as a guide to allow the first or second leg 102, 104 to slide down a guide wire to a bony surface. The first and second elongated slots 118, 120 may be adapted to allow the legs to move relative to one another without disturbing or moving the guide wire. In certain embodiments, each foot may include a surface adapted to be placed adjacent a fastener head or a bony surface.

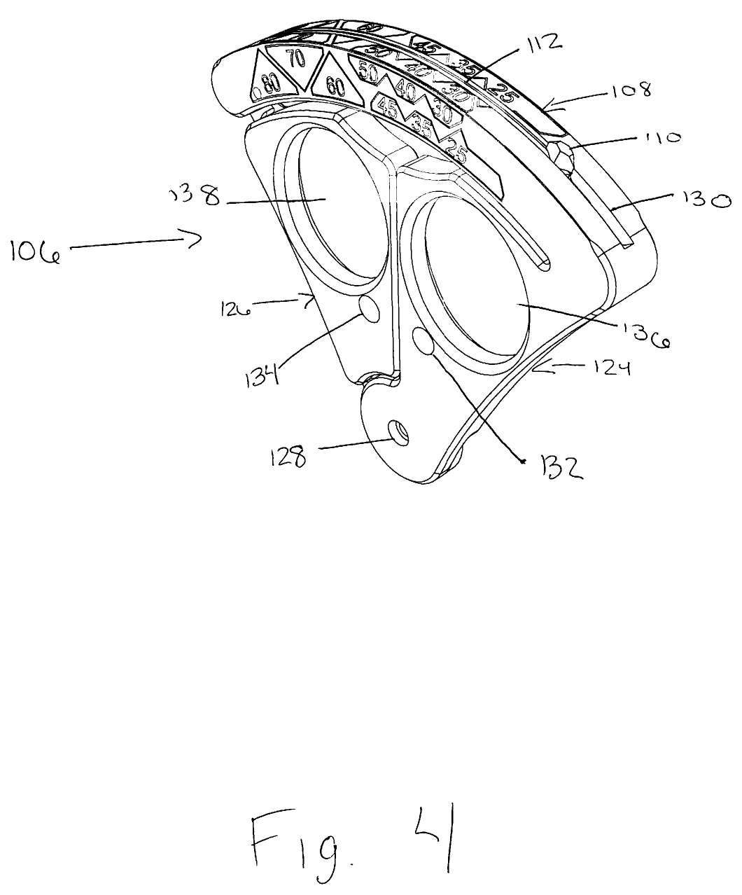

In FIG. 4, an enlarged view of one embodiment of the proportional magnifier 106 is shown. In some embodiments, the proportional magnifier 106 may include a first arm 124 and a second arm 126. The first and second arms 124, 126 may be pivotally coupled together about a pivot point 128. The pivot point 128 may comprise a fastener or a pin about which the arms may pivot. The proximal end portion of the first arm 124 may be attached to a scale arm 108. Each side of the scale arm may include a plurality of markings that may correspond to the distance between the first and second feet 114, 116. In some embodiments up to three sides of the scale arm 108 may be marked with a plurality of markings so that a surgeon may be able to read the measured distance from more than one direction. The plurality of markings may directly correspond to an actual measurement between the first and second foot 114, 116. The distance may be marked by an indicator 110 that may be attached to an indicator arm 112. The indicator 110 may move along an elongated opening 130 as the first and second feet 114, 116 are moved relative to each other.

In some embodiments, the first arm 124 may be connected to the first leg 102 about a pivot point 132 and the second arm 126 may be connected to the second leg 104 about a pivot point 134. The attachment of the first and second arms 124, 126 to the first and second legs 102, 104 and to each other, may allow the first and second arms 124, 126 to amplify the angle between the first and second legs 102, 104. The amplified angle may be evidenced by a larger angle between the first and second arms 124, 126 and a larger scale arm 108. This amplified angle and larger scale arm 108 may allow a surgeon to more accurately determine a correct measurement between first and second feet 114, 116.

In certain embodiments the first arm 124 may include a first finger hole 136 and the second arm 126 may include a second finger hole 138. The first and second finger holes 136, 138 may be used by a surgeon to move the first and second arms 124, 126 relative to each other about pivot point 128. In certain embodiments, as the first and second arm 124, 126 move relative to each other, the first and second legs 102, 104 may also move relative to each other about pivot point 140 (FIG. 1). As the first and second leg 102, 104 pivot, the first and second feet 114, 116 may also move relative to one another. The distance between the feet 114, 116 may be determined by the location of the indicator 110 on the scale arm 108. The distance between the feet 114, 116 may be used to determine the size of an implant to be implanted.

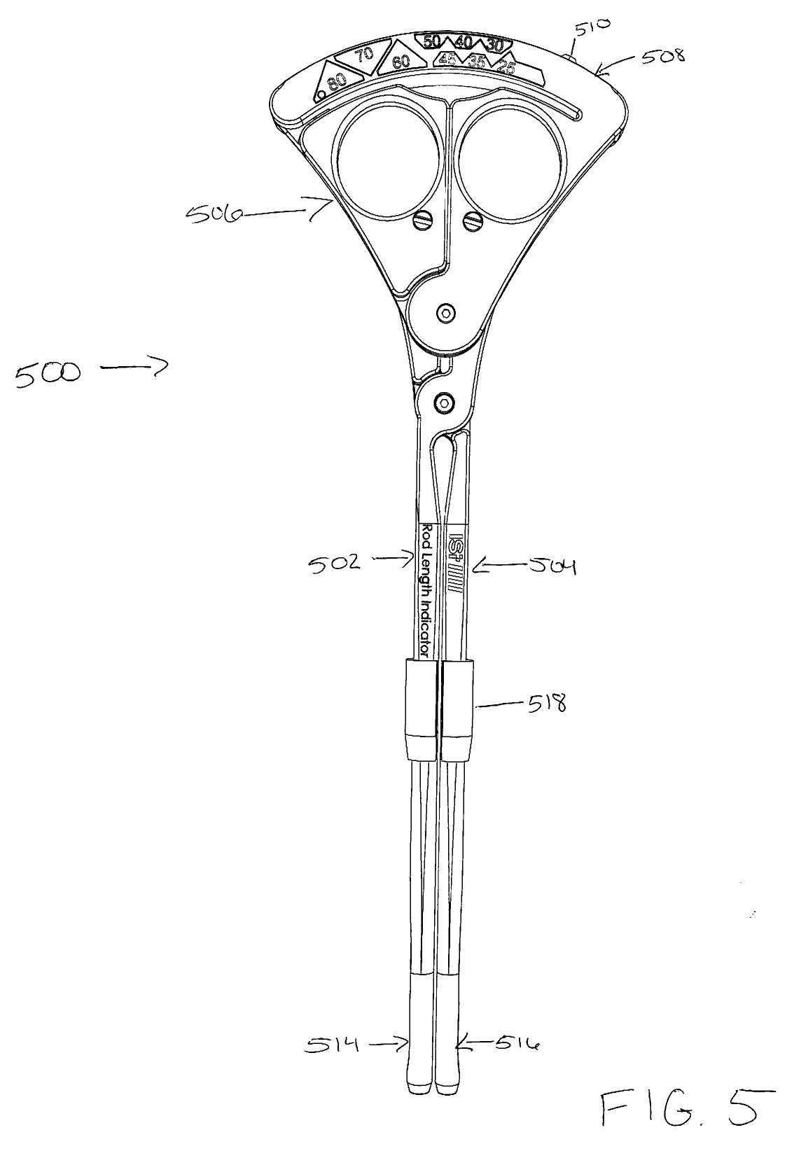

Referring now to FIG. 5, another embodiment of a measuring device 500 is shown that may be used to measure the distance between two bony structures. The measuring device 500 may include a first leg 502 and a second leg 504 coupled to each other. In certain embodiments the first and second legs 502, 504 are pivotally coupled to each other. The distal end portion or feet 514, 516 of the first and second legs 502, 504 may be adapted to be placed adjacent a bony structure or a device that may be implanted into the bony structure. The proximal end portions of the first and second legs 502, 504 may be coupled to a proportional magnifier 506. The proportional magnifier 506 may include a scale arm 508 with an indicator 510 that indicates to a user of the measuring device the distance between the bony structures that may be adjacent the distal end of the first and second feet 514, 516. The proportional magnifier may work similarly to the proportional magnifier discussed with regards to FIG. 4.

In certain embodiments, the measuring device 500 may include a centering bushing 518. The centering bushing 518 may allow a surgeon to more accurately guide a leg down an extension that may be attached to an implant in the bony structure. The centering bushing may allow for more accurate placement of the feet 514, 516 relative to an implanted structure by centering the leg over an implanted structure.

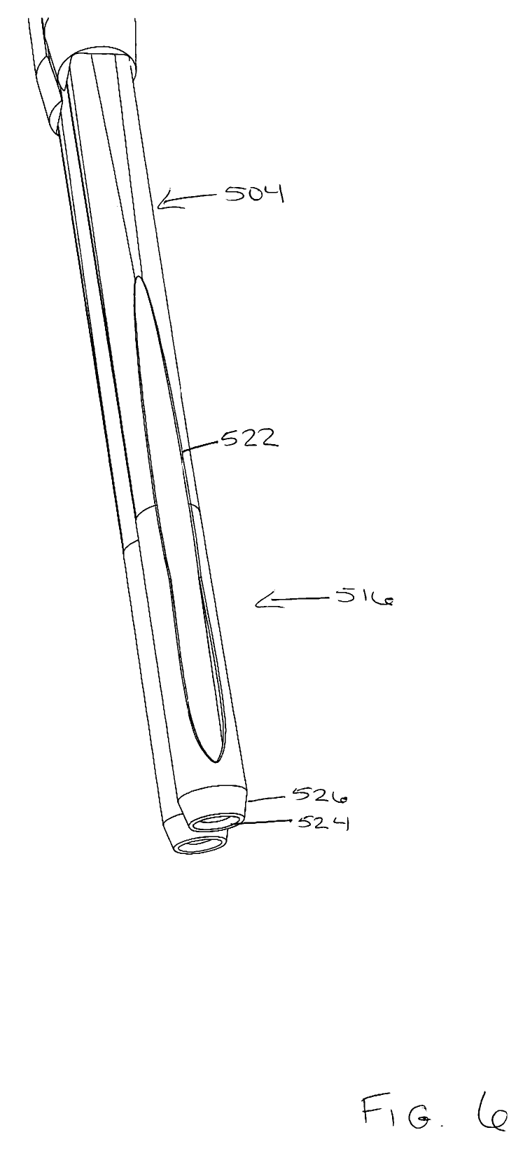

In FIG. 6, an enlarged view of the side of distal end portion of leg 504 including second foot 516 is shown. The side of the foot 516 may include an elongated slot 522 and a distal aperture 524. The distal aperture 524 allows the second leg 504 of the measuring device 500 to slide down over a guide wire. The elongated slot 522 may allow the guide wire to extend out the side of the device. The foot 516 may also have a surface portion 526 that may be adapted to be placed along side a bony structure or adjacent an implant. Although only second foot 516 is illustrated, the first foot 514 may have similar features.

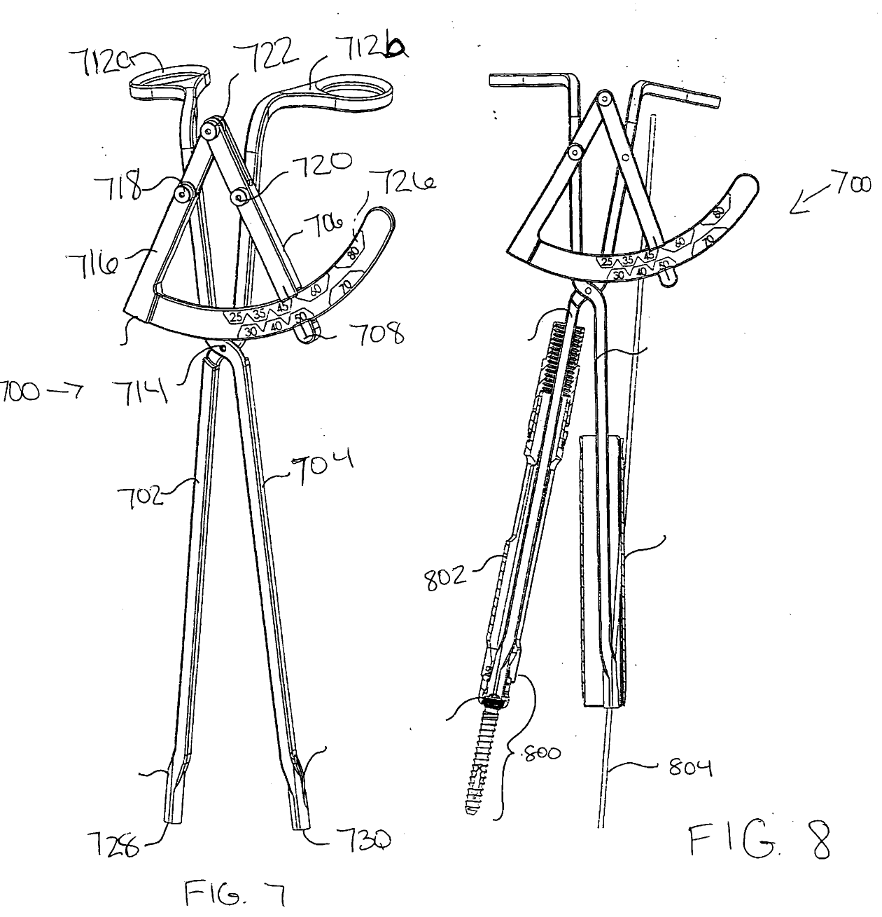

Referring now to FIG. 7, one embodiment of a measuring device 700 is illustrated that may be used to measure the distance between two bony structures. The measuring device 700 may have legs 702 and 704 and an indicator arm 706 that may move in relation to a scale arm 726. The scale arm 726 may have the actual measurements thereon. Indicator arm 706 may have indictor 708 thereon showing the distance between screws displayed in a plurality of markings on the scale arm. The device 700 may include a handle 712a that may be an extension to leg 704 and also may have a bend for finger insertion. Leg 702 may include a handle 712b.

In certain embodiments, as the handles move apart so do the legs, pivoting around a pin 714. A fixed portion 716 may pivot around a pin 718 connected to leg 704 while indicator arm 706 pivots around a pin 720 attached to leg 702. Both parts then may pivot about a pin 722 so that as the distal ends of the legs may separate from one another. The legs 704, 702 may pivot about pins 714, 722 causing the indicator arm 706 to move across the path of the radius of the arc between the pedicle screws. The radius in this case being the length from pin 722 to the numbers on measuring arm 726. This then may allow a distance to be determined at the distal end of the tool. The plurality of markings on scale arm 726 is adjusted to account for the variance between the implanted pedicle screw and the arm. In the illustrated embodiment, the proportional magnifier 730 is not integrated into the handle. Thus the scale arm 726 may be towards the distal portion of the instrument.

Tool 700 has two openings 728 and 730 at the bottom of legs 702 and 704, respectively. These openings are adapted to engage whatever features they are to measure the distance between. This measurement tool would be typically used once one screw is positioned. Also, measurements can be taken across two guide wires between pedicles so that a rod length can be selected.

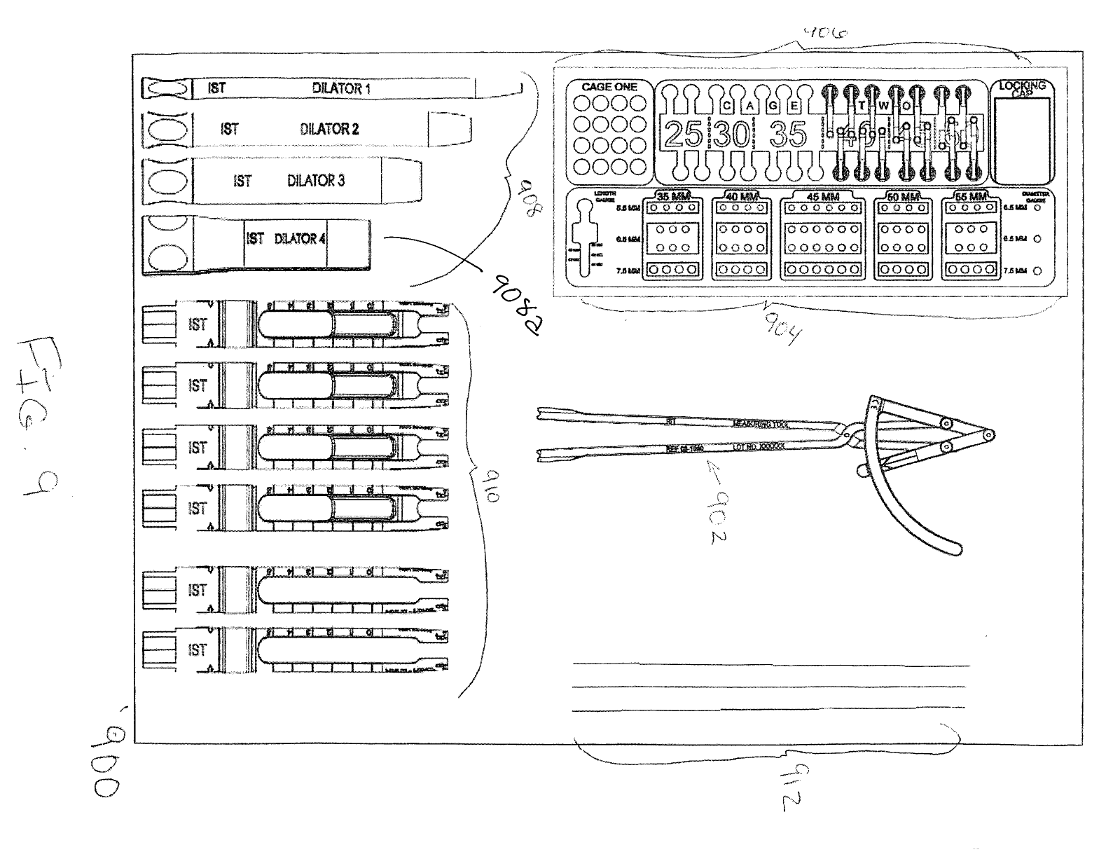

Referring now to FIG. 8, one embodiment of the measuring device 700 is shown during use. During a subcutaneous surgical procedure for implanting a spine stabilization system between adjacent vertebrae, bone anchors such as pedicle screws are generally placed near the beginning of the procedure. For example a first pedicle screw assembly 800 is placed into a first vertebra with an extension 802 extending therefrom for placing remaining components of the stabilization system. A guide wire 804 may be run down to a second vertebra that does not yet have a pedicle screw implanted therein. The distance between the first pedicle screw and the point on the second vertebra wherein a second pedicle screw will be placed may need to be measured in order to determine the proper sizing of a stabilizing rod to be placed between the two vertebrae. The distal end of tool 700 may come to a rest on top of the implanted screw 800 and may mate with a driving portion of the implanted screw 800. The second leg 704 may then be positioned over a guide wire 804 and slipped down the guide wire to the base of the pedicle. The indicator 708 may be positioned along a specific point relative to the plurality of markings on indicator arm 706, thereby indicating the distance between the two points. This then may allow the surgeon to read the pedicle to pedicle distance on the tool. The measurement of the distance may be taken by reading the nearest marking of located to the point at which the indicator 708 rests The measurement tool can also be used to measure cross connector lengths, or another distance within the limits of the scale of the measurement tool.

In FIG. 9, an embodiment of a kit 900 for implanting a stabilization device into the spine is shown. The kit 900 may include a measuring instrument 902, a plurality of pedicle screws 904 adapted to be placed in a patient's vertebra, a plurality of implants 906 adapted to be attached to the pedicle screws and placed adjacent a patient's spine, a plurality of dilator 908, where at least one of the dilators 908a having an elongated slot for expanding an opening in the patient's tissue, a plurality of extensions 910 adapted to be attached to the plurality of pedicle screws, wherein the extensions may include elongated openings, and a plurality of guide wires 912 to guide implants and tools to the surface of a vertebra. Details of these instruments can be found in U.S. application Ser. No. 10/990,272, previously incorporated by reference.

The forgoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

For instance, in some embodiments, there may be an instrument for measuring the distance between two bony structures comprising at least two legs having proximal and distal ends pivotally mounted about a fastener, handles coupled to the proximal ends of the at least two legs, the handles comprising a first and second indicator arm which move about a common axis as the handles move apart, an indicator on one end of the first indicator arm, and measurement markings on the second indicator arm.

In yet other embodiments, there may be an instrument for measuring the distance between two bony structures further comprising feet on the distal ends of the legs for engaging points on the bony structures. In still other embodiments, the feet may further comprise indentions therein.

In other embodiments, there may be an instrument for measuring the distance between two bony structures comprising at least two legs having proximal and distal ends pivotally mounted about a fastener wherein the fastener comprises a pin. In still other embodiments the handles may be coupled to the legs by pins.

In another embodiment, the handles may comprise openings for receiving fingers of a user of the instrument. In still other embodiments, the measurement markings on the second indicator arm may comprise numerals representing certain lengths.

In other embodiments, there may be an instrument for measuring the distance between two bony structures comprising at least two legs having proximal and distal ends pivotally mounted about a fastener, the legs comprising feet on the distal ends of the legs, wherein the feet comprise indentions therein, handles coupled to the proximal ends of the at least two legs, the handles comprising a first and second indicator arm which move about a common axis as the handles move apart, and openings therein for receiving fingers of a user of the instrument, an indicator on one end of the first indicator arm, and measurement markings comprising numerals on the second indicator arm.

What is claimed is:

1. A kit for use in spinal stabilization procedures comprising:
an instrument for measuring the distance between two bony structures;
a plurality of rods for spanning the distance between the two bony structures;
a plurality of pedicle screws to be placed into the bony structures;
a plurality of guide wires to guide a pedicle screw into place in the bony structure.

2. The kit of claim 1, further comprising a dilator having a longitudinal slot.

3. The kit of claim 1, wherein the plurality of pedicle screws are of different sizes.

4. The kit of claim 1, wherein the plurality of rods comprises rods of different sizes.

5. The kit of claim 1 further comprising a plurality of extensions.

6. A medical instrument for measuring, comprising:
a handle portion;
a first leg having a distal end portion and a proximal end portion and a second leg having a distal end portion and a proximal end portion;
a proportional magnifier coupled to the first and second leg, wherein the proportional magnifier has a first arm and a second arm; and
a scale coupled to the first and second arm of the proportional magnifier.

7. The medical instrument of claim 6 wherein the first leg and the second leg intersect at an intersection point in between the distal and proximal end portion of each leg.

8. The medical instrument of claim 7 wherein the first and second legs are pivotally coupled at the intersection point.

9. The medical instrument of claim 6 wherein the first leg has a distal end, wherein the distal end has a slot.

10. The medical instrument of claim 9 wherein the first leg has an aperture in the distal end.

11. The medical instrument of claim 6 further comprising a handle portion.

12. The medical instrument of claim 11 wherein the handle portion is located proximal to the proportional magnifier.

13. The medical instrument of claim 11 wherein the handle portion is located distally of the scale.

14. The medical instrument of claim 6 wherein the scale has a plurality of markings that directly corresponds to a distance between the distal end of the first and second legs.

15. The medical instrument of claim 6 wherein the proportional magnifier comprises a first arm having a proximal end and a distal end and a second arm having a proximal end and a distal end.

16. The medical instrument of claim 15 wherein the proximal end of the first arm and the proximal end of the second arm are pivotally coupled to each other.

17. The medical instrument of claim 16 wherein the first arm is pivotally connected to the first leg and the second arm is pivotally connected to the second leg.

18. The medical instrument of claim 16 wherein the distal end of the first arm is attached to the scale.

19. The medical instrument of claim 16 wherein the distal end of the second arm has an indicator that is directly coupled to the scale.

20. The medical instrument of claim 15 wherein the distal end of the first arm and the distal end of the second arm are pivotally coupled to each other.

21. The medical instrument of claim 20 wherein the first leg has a proximal end and the second leg has a proximal end, wherein the proximal end of the first leg is pivotally coupled to the first arm and the proximal end of the second leg is pivotally coupled to the second arm.

22. The medical instrument of claim 21 wherein the proximal end of the first arm is attached to the scale.

23. The medical instrument of claim 22 wherein the proximal end of the second arm is attached to an indicator arm.

24. The medical instrument of claim 23 wherein the indicator arm has an indicator capable of aligning with a marking from a plurality of markings on the scale.

25. The medical instrument of claim 24 wherein the indicator arm is slidingly coupled to the scale.

26. A method of using a medical instrument for measuring the distance between bony structures, comprising:
implanting a first pedicle screw into a first vertebra, wherein the implanted first pedicle screw is coupled to an extension;
placing a guide wire in a second vertebra to mark the location of placement for a second pedicle screw;
providing an instrument for measuring the distance, comprising: a first leg and a second leg pivotally coupled to each other, a proportional magnifier coupled to the first and second leg, a scale coupled to the proportional magnifier, and a handle portion coupled to the first and second legs;
gripping the handle portion;
sliding the first leg down the extension to a top of the first pedicle screw;
sliding the second leg down the guide wire to a top of the vertebra;
releasing the handle portion;
reading the scale to determine a distance between the first and second vertebra, wherein the distance is amplified by the proportional magnifier to be readable by a surgeon;
selecting an implant that corresponds to the distance between the first and second vertebra.
